(19) United States
(12) Patent Application Publication
(10) Pub. No.: US 2014/0087059 A1
(43) Pub. Date: Mar. 27, 2014

(54) PHARMACEUTICAL COMPOSITION AND PROCESS FOR MONTELUKAST TABLETS

(71) Applicant: PHARMA PASS LLC, Irvine, CA (US)

(72) Inventors: Benoit Schmitt, Irvine, CA (US); Laurent Imler, Irvine, CA (US)

(73) Assignee: PHARMA PASS LLC, Irvine, CA (US)

(21) Appl. No.: 13/624,227

(22) Filed: Sep. 21, 2012

(51) Int. Cl. A61K 31/47; A61P 11/08; B05D 7/00; A61P 11/06

(52) U.S. Cl. 427/2.14; 514/311

(57) ABSTRACT

The manufacture of compositions containing montelukast and to stable tablet compositions resulting thereof are disclosed, which include a first compaction step of a dry blend including, montelukast or a pharmaceutically acceptable salt thereof, and microcrystalline cellulose, and a further compression step into tablets.

### FIELD OF THE INVENTION

The present invention relates to a novel process for the manufacture of compositions containing montelukast and to stable tablet compositions comprising montelukast.

### BACKGROUND

Montelukast sodium is a quinoline compound which is described chemically as [R-(E)-1-[[[1-[3-[2-[7-chloro-2-quinolinyl]ethenyl]phenyl]-3-[2-(1-hydroxy-1-methylethyl)phenyl]propyl]thio]methyl]cyclopropane acetic acid and can be represented by the following chemical formula.

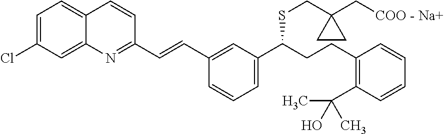

A therapeutical value was attributed to this compound which seems to work as a leukotriene receptor antagonist that can be used in the treatment or the prevention of symptoms induced by the leukotrienes such as pulmonary disorders. Montelukast has particularly proved to be useful in the treatment or prevention of asthma, chronic bronchitis and related obstructive airways diseases. The basic U.S. Pat. No. 5,565,473 discloses these indications. It is an exceptionally labile agent which exhibits rapid degradation in the presence ambient conditions such as UV light, heat and particularly degrades once in contact with water or moistures.

Montelukast is currently marketed under the tradename Singulair® in the form of a chewable 5 mg tablet and of a 10 mg coated tablet for the treatment of asthma. Both of these formulations contain in addition to montelukast sodium, hyprolose, microcrystalline cellulose, croscarmellose sodium, lactose or mannitol and magnesium stearate. Unfortunately, these formulations suffer from a rapid degradation of the active ingredient which requires that the tablets are administered rapidly once they are taken out of the containers and results in low patient compliance. Thus there exists a need to develop formulations and processes which provides stabilized formulations of montelukast.

U.S. Pat. No. 5,565,473 discloses that pharmaceutical compositions containing montelukast may be presented as a compressed tablet which is prepared by a conventional method consisting in compressing in a suitable machine, the active ingredient in a free flowing form such as powder or granules, optionally mixed with a binder, lubricant, inert diluent, surface active or dispersing agent. Nevertheless, the process disclosed in U.S. Pat. No. 5,565,473 does not comprise a preliminary compaction step.

U.S. Patent Publication No. 2007/0184101 is specifically concerned with stable formulations containing montelukast. This document particularly alleges that the loss in stability is attributed to the presence of microcrystalline cellulose which should thus be avoided in the composition of tablets or capsules. Nevertheless, applicant has surprisingly discovered that compositions comprising microcrystalline cellulose could provide stable tablet forms if subjected to a preliminary compaction together with the active ingredient.

U.S. Patent Publication No. 2009/0247575 is also concerned with the provision of a stable formulation of montelukast. However, the document, which does not disclose a manufacturing process comprising a compaction step teaches that montelukast should be formulated with a stabilizing vehicle comprising liquid triglycerides and a desiccant.

WO 2007/077135 discloses dry processes for the preparation of tablets of montelukast which nevertheless do not imply an intermediate compaction of the powder or granules containing the active ingredient before the final compression into tablet forms. This document does not either disclose tablets of montelukast with sucralose.

### SUMMARY

The invention is related to a process for the preparation of tablets comprising a first compaction step of a dry blend comprising montelukast or a pharmaceutically acceptable salt thereof and microcrystalline cellulose and a further compression step into tablets. Preferred embodiments comprise one or more of the following features:

The compacted blend is granulated and the resulting granules are compressed into tablets.
Additional excipients are added to the compacted blend before compression.
The tablets are further coated.
The additional excipients are selected among disintegrants, lubricants or glidants.
The additional excipients are selected among colloidal silicon dioxide, starch, magnesium stearate, sodium stearyl fumarate, croscarmellose sodium, crospovidone, sodium starch glycolate and talc.
The dry blend further comprises sucralose.
The compaction step is performed using a roll compactor.
The compression step is performed suing a rotating press.
The compaction step is performed at a pressure within the range of 2 to 4 Mpa, for example 3 MPa.
The compaction is performed at a powder flow of 5 to 20 g/min, for example about 11 g/min.
The invention is also related to a tablet obtainable by the above-disclosed process.

Preferred embodiments comprise one or more of the following features:

The tablet is of a chewable type.
The tablet comprises 1 to 20 mg of montelukast or of a pharmaceutically acceptable salt thereof, for example 5 or 10 mg.
The tablet further comprises a binder and/or a diluent and/or a glidant and/or a disintegrant.
The binder is any selected among acacia, alginic acid, carbomer, sodium carboxymethylcellulose, dextrin, ethylcellulose, gelatine, glucose, guar gum, hydroxypropylcellulose, maltose, methylcellulose, povidone, polyvinylpyrrolidone, starch, methylcellulose or polyethylene oxide, the diluent is any selected among microcrystalline cellulose, calcium, phosphate or sulfate carbonates, dextrates, dextrins, dextrose excipients, fructose, kaolin, lactitol, anhydrous lactose, lactose monohydrate, maltose, mannitol, sorbitol, sucrose, starch, pregelatinized starch, or talc, the glidant is any selected among: colloidal silicon, magnesium trisilicate, starch, talc or tribasic calcium phosphate and the disintegrant is any selected among: alginic acid, croscarmellose sodium, crospovidone, potassium polacrilin, sodium starch glycolate, and starch.

A tablet having the following composition:

| | | |
|---|---|---|
| Montelukast sodium | 5.20 | mg |
| Hydroxypropyl cellulose | 35.00 | mg |
| Microcrystalline cellulose | 10.00 | mg |
| Mannitol | 195.00 | mg |
| Flavour | 3.60 | mg |
| Iron oxide red | 0.10 | mg |
| Sodium stearyl fumarate | 3.50 | mg |
| Sucralose | 0.50 | mg |
| Aerosil | 0.90 | mg |
| (AcDiSol) Crossed linked sodium | 10.00 | mg |
| carboxymethyl cellulose | | |
| Sodium stearyl fumarate | 2.80 | mg |
| Total | 266.60 | mg |

A tablet having the following composition:

| | | | |
|---|---|---|---|
| Tablet | Montelukast sodium | 10.40 | mg |
| | Hydroxypropyl cellulose | 20.00 | mg |
| | Microcrystalline cellulose | 10.00 | mg |
| | Mannitol | 120.00 | mg |
| | Aerosil | 0.80 | mg |
| | (AcDiSol) Crossed linked sodium | 20.00 | mg |
| | carboxymethyl cellulose | | |
| | Sodium stearyl fumarate | 1.80 | mg |
| | Total | 185.00 | mg |
| Coating | Opadry II 85 F white | 5.50 | mg |

| | |
|---|---|
| Total | 190.50 |

A tablet comprising montelukast or a salt thereof, microcrystalline cellulose and sucralose wherein the amount of impurity does not increase by more than 0.4% by weight from the initial amount of montelukast after storage at about 40° C. and about 75% relative humidity for 1 month, for example 0.2%.

The invention is also related to a method for treating or preventing a leukotriene induced condition, comprising the step of administering orally to a patient in need thereof, a tablet as above disclosed. Preferred embodiments comprise one or more of the following features:

The leukotriene induced condition is asthma or chronic bronchitis.

### BRIEF DESCRIPTION OF THE DRAWINGS

FIG. 1 represents the results of the stability test performed in Example 1 under accelerated storage conditions at 40° C. and 75% relative humidity;

FIG. 2 represents the results of the stability test performed in Example 2 under accelerated storage conditions at 40° C. and 75% relative humidity;

FIG. 3 represents the dissolution profile performed in Example 1 in phosphate buffer pH 6.8; and

FIG. 4 represents the dissolution profile performed in Example 2 in phosphate buffer pH 6.8.

### DETAILED DESCRIPTION

The present invention relies on the surprising finding that tablet compositions of montelukast having an improved stability could be obtained from a dry process comprising a first compaction of a dry blend comprising the active ingredient together with a binder which is microcrystalline cellulose before a compression into tablet forms. Wet granulation processes are usually preferred over dry compression techniques for the preparation of tablets. For example when the amount of an active substance contained in the tablet is low one may foresee difficulties in dispersing the drug uniformly in the powder blend using a conventional direct compression process. In addition, wet granulation processes are usually preferred for the preparation of chewable tablets. For example, the dispersion or the size of the granules obtained through the amount of wetting agent involved in the granulation usually influences the organoleptic qualities of the final product. Thus, dry techniques are more challenging as it seems more difficult to meet all the characteristics of free-flowing mixtures of the dry blend and of homogeneity. In the present case, a dry compression process has notably the advantage over any conventional wet granulation process that the use of water and/or other wetting agents is avoided, which increases the risk of degradation of montelukast.

The process according to the invention may be performed by blending a first composition containing montelukast together with a microcrystalline cellulose binder and optionally with other excipients. For example, the use of diluents may be advantageous to increase the bulk of the solid pharmaceutical composition. Excipients include in a non limiting way, binders, diluents, glidants, disintegrants, sweetener or flavoring agents.

The resulting blend is then compacted into a slug or a sheet which can be subsequently sieved into compacted granules. This step has proved to be particularly advantageous in the preparation of stable compositions of montelukast, and the microcrystalline binder which is compacted in the surrounding of the active ingredient seems to protect it from degradation. It may also be advantageous to first blend the active ingredient together with the microcrystalline binder and at least part of the diluent before compaction.

One appropriate sieving method involves passing a powder through a mesh of defined size in order to exclude particles below the specified size. Air may concomitantly be used to carry away the fine particles.

The operating conditions for compaction will be those that are available to the skilled man. Within the scope of the present invention, the blend can be compacted by either slugging or passing the material between two counter-rotating rolls. The compaction force may be adjusted using a method appropriate for the compactor employed, for example by control of the rate of feed into the compactor.

The compacted granules thus obtained may subsequently be compressed into a tablet, typically with the addition of a lubricant or of other excipients. The tablets preferably have a crushing strength in the range of 25 to 170 N, for example 30 to 140 N. These can be determined by standard techniques on a Erweka Multicheck tester.

The compressed tablet may further be coated with a coating agent to form film coated tablets. The coating agent may be suspended in a solution or directly taken from a commercially available coating solution which is sprayed onto the compressed pellets. Preferably, all process steps are carried out in controlled atmosphere, such as low moisture, oxygen, temperature and light protection.

The present invention is also concerned with the resulting tablets thus obtained. These exhibit an improved stability as shown in the tests performed in the examples below which is attributed to the specific method of preparation wherein a prior compaction is performed before any compression step.

In the tablets of the present invention, montelukast can be in its acid form or any of its pharmaceutically acceptable salts, solvates or polymorphs. Typically, montelukast may be used in the form of an alkali metal salt, such as its sodium salt. In addition, montelukast may be present in a micronized form, in order to improve the dispersion of the drug into the dry blend before the preliminary compaction. The term “micronized” within the meaning of the present application refer to a material having a particle size ranging from about 1 to 30 μm.

In the framework of this invention, binders refer to excipients which enhance the linkage between particles. They include in a non limiting manner, any of acacia, alginic acid, carbomer, sodium carboxymethylcellulose, dextrin, ethylcellulose, gelatine, glucose, guar gum, hydroxypropylcellulose, maltose, methylcellulose, povidone, polyvinylpyrrolidone, starch, methylcellulose or polyethylene oxide. Advantageously, the binder involved in the process of the present invention and which comes into the composition of the final tablets is hydroxypropylcellulose (HPC), such as low substituted hydroxypropylcellulose.

Typically, the amount of binder within the scope of the invention is comprised, based on the total amount of the tablet, within the scope of 1 to 20% by weight, for example 5 to 15% by weight, i.e. about 10% by weight. Diluents intend to increase the bulk of the composition in order to facilitate the processing of tablets comprising low amounts of active ingredients such as in the present case with low therapeutical amounts of montelukast. Diluents within the scope of the invention comprise in addition to microcrystalline cellulose, calcium, phosphate or sulfate carbonates, dextrates, dextrins, dextrose excipients, fructose, kaolin, lactitol, anhydrous lactose, lactose monohydrate, maltose, mannitol, sorbitol, sucrose, starch, pregelatinized starch, or talc. Typically, the diluent represent about 50 to about 95% by weight based on the total weight of the tablet, for example 60 to 90% by weight, i.e. about 75% by weight.

The tablets according to the invention, which may be of a chewable type, may further comprise sweetening agents. While tablets of the prior art usually involves aspartame, the sweetener comprised within the scope of the invention may be, without being limited to aspartame, dextrates, dextrose, fructose, saccharin, sorbitol, sucralose, sucrose, sugar, or syrups. Non calorific excipients are preferred since they usually have a stronger sweetening effect than saccharose derivatives which allows decreasing the overall bulk amount within the final tablet.

In addition, it has been surprisingly discovered that the use of sucralose could further improve the stability of the final tablets when compacted with the active ingredients and microcrystalline cellulose according to the above process. Advantageously, the sweetening agents may represent 0.01 to 1% by weight based on the total weight of the tablet, for example 0.05 to 0.3% by weight, i.e. about 0.2% by weight.

In addition, the tablets of the invention may further comprise flavoring agents to improve its compliance in patients. These include without being limited to, maltol, vanillin, ethyl vanillin, menthol, citric acid, fumaric acid, ethyl maltol, tartaric acid, peppermint, fruit flavours, natural or artificial flavours such as orange flavour. Advantageously, the flavouring agents represent 0.1 to 3% by weight based on the total weight of the tablet, for example 1 to 2% by weight, i.e. about 1% by weight. These agents are particularly suited for a chewable type tablet.

The tablets according to the invention may further comprise disintegrants, typically in an amount comprised within the range of 0.1 to 10% by weight of the total composition, for example 1 to 5% by weight, i.e. about 4% by weight. Without being restrictive, the disintegrants within the scope of the invention include, alginic acid, croscarmellose sodium, crospovidone, potassium polacrilin, sodium starch glycolate, and starch. Advantageously, the AcDiSol®disintegrant provides significant results in the dissolution rate of a tablet into the body after administration.

Lubricants are usually useful to prevent adhesion during the preparation process. These are of particular use for the process of the invention which implies the preliminary compaction of powders and/or granules before the final compression into a tablet. Suitable lubricants are, in a non limiting manner, calcium stearate, glyceryl behenate, magnesium stearate, mineral oil, polyethylene glycol, sodium stearyl fumarate, stearic acid, talc, vegetal oil, sodium lauryl sulfate or zinc stearate. Lubricants may advantageously be incorporated into the composition of the tablets of the invention in an amount comprised within the range of 0.1 to 5% by weight based on the total weight of the tablet, for example 1 to 3% by weight, i.e. about 2%.

Glidants may be useful in the early stages of the process of the invention in order to improve the flowability of the powder/granules before the compaction step. Thus, glidants may come into the composition of the tablets of the invention. Suitable glidants within the scope of the invention are, in a non limiting manner, colloidal silicon, magnesium trisilicate, starch, talc or tribasic calcium phosphate. Glidants may advantageously be incorporated into the composition of the tablets of the invention in an amount comprised within the range of 0.1 to 3% by weight based on the total weight of the tablet, for example 1 to 2% by weight, i.e. about 1%.

The coating agent can be made from any commercially available powder mix for preparing coating suspensions. Examples of such powders or mix are, Opagloss® or Opadry® available from Colorcon, which comprises hydroxypropyl cellulose, hypromellose, titanium dioxide and iron oxide. Examples of Opadry® are Opadry® II HP, Opadry® 20A, or Opadry® white. The coating may be prepared from the individual elements rather than from the commercially available preparation. Coating agents may advantageously represent 0.1 to 5% by weight based on the total weight of the tablet, for example 1 to 5% by weight, i.e. 3% by weight. Further excipients are disclosed in Handbook of Pharmaceutical excipients, 2^{nd} Ed., 1994, American Pharmaceutical Association, Washington, ISBN 0 91730 66 8, by Wade A., Weller P J.).

### EXAMPLES

### Example 1

### Preparation of Tablets According to the Invention

Two tablets having the following composition (a chewable and a coated tablet) were prepared.

| | | |
|---|---|---|
| Montelukast sodium | 5.20 | mg |
| Hydroxypropyl cellulose | 35.00 | mg |
| Microcrystalline cellulose | 10.00 | mg |
| Mannitol | 195.00 | mg |
| Flavour | 3.60 | mg |
| Iron oxide red | 0.10 | mg |
| Sodium stearyl fumarate | 3.50 | mg |
| Sucralose | 0.50 | mg |
| Aerosil | 0.90 | mg |
| (AcDiSol) Crossed linked sodium | 10.00 | mg |
| carboxymethyl cellulose | | |
| Sodium stearyl fumarate | 2.80 | mg |
| Total | 266.60 | mg |

| | | | |
|---|---|---|---|
| Tablet | Montelukast sodium | 10.40 | mg |
| | Hydroxypropyl cellulose | 20.00 | mg |
| | Microcrystalline cellulose | 10.00 | mg |
| | Mannitol | 120.00 | mg |
| | Aerosil | 0.80 | mg |
| | (AcDiSol) Crossed linked sodium | 20.00 | mg |
| | carboxymethyl cellulose | | |
| | Sodium stearyl fumarate | 1.80 | mg |
| | Total | 185.00 | mg |
| Coating | Opadry II 85 F white | 5.50 | mg |

| | |
|---|---|
| Total | 190.50 |

First, the amount of Montelukast sodium and microcrystalline cellulose were mixed together with hydroxypropyl cellulose, mannitol, and sodium stearyl fumarate before being passed through a 0.710 mm screen. The first composition (5 mg) further comprised sucralose, the flavours and pigments. The blend was mixed for 10 minutes in Turbula T2C before being compacted on a roller compactor TFC Labo, Vector Corp. The roll applied a pressure of 3 Mpa at a roll speed of 3-4 rpm and a powder flow of 11 g/min.

The resulting compacted sheet was passed through a Vector Corp. rotating granulator (at 160 rpm) with 18 mesh screen. After having weighted the appropriate amount of the compacted granules for the final composition, sodium stearyl, aerosol and Acdisol® were added to the mixture and blended for 10 minutes in the Turbula. The resulting mix was finally compressed on a Manesty Betapress type rotating press respectively using a 9 and 8 mm round tooling. The second composition (10 mg) was passed through a 0.710 mm screen before the final blending and compression.

The 10 mg tablets were further coated by suspending Opadry® in water, thus providing a suspension that was stirred for 45 minutes. The resulting suspension was then applied to the tablet using a 1 mm nozzle (distance nozzle-tablet bed: 8-9 cm; inlet temperature 50-60° C.; product temperature 39-41° C.; air flow 45-65° cfm; solution flow 6-9 g/min, spraying pressure 25 PSi and Pan speed: 15 rpm) and dried for 30 minutes at 45° C. The resulting tablets showed a thickness of about 4.55 mm and a hardness of 6.0-7.0 kP using a hardness tester Schleuniger 4M for the chewable tablet, and a thickness of about 4.05 mm for the coated tablet.

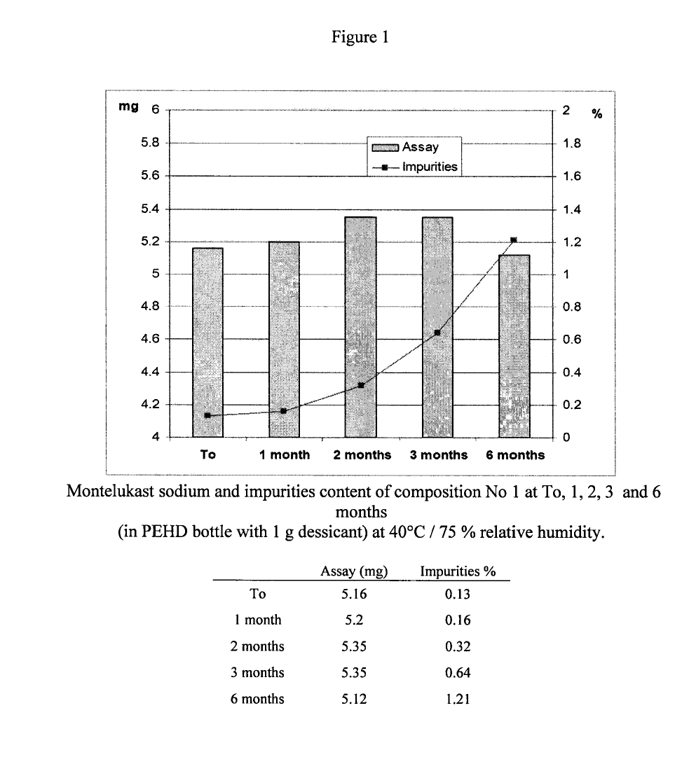

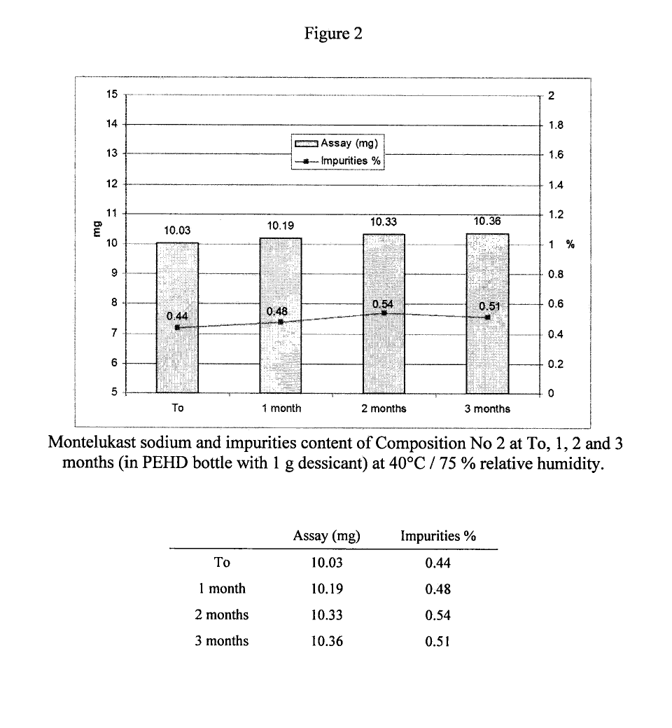

These two tablet compositions have been subjected to accelerated degradation at 40° C. and 75% relative humidity to assess their stability. The results, provided in the tables of FIGS. 1 and 2 illustrate the improved stability of the tablets according to the invention. In comparison, the existing Singulair® tablets respectively exhibited for the same amount of Montelukast 0.2 and 0.49% impurities after one month and under the accelerated storage conditions.

The 5 mg tablet exhibit an amount of impurity which increases moderately, reaching a value below 2% of montelukast content after 6 months in stressed conditions. The 10 mg tablet exhibits even improved stability with values around 0.5% after 3 months in stressed conditions. These results are illustrative of very stable compositions.

### Example 2

### Dissolution Tests

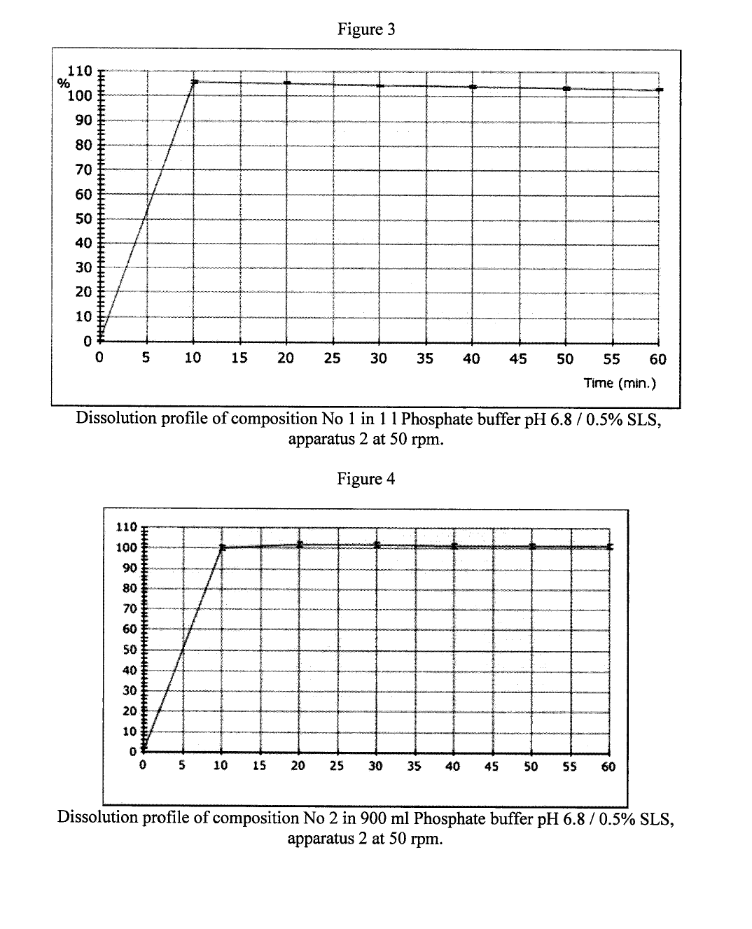

The two previous tablets prepared according to example 1 were dissolved at pH 6.8, using USP phosphate buffer with 0.5% (w/w) SLS (Sodium Lauryl Sulfate), and Apparatus 2 (paddles) at 50 rpm, respectively in 1000 and 900 ml of buffer. The dissolution profiles are illustrated in FIGS. 3 and 4.

1. A process for the preparation of tablets comprising a first compaction step of a dry blend comprising montelukast or a pharmaceutically acceptable salt thereof and microcrystalline cellulose and a further compression step into tablets.

2. The process according to claim 1, wherein the compacted blend is granulated and the resulting granules are compressed into tablets.

3. The process according to claim 1, wherein additional excipients are added to the compacted blend before compression.

4. The process according to claim 1, wherein the tablets are further coated.

5. The process according to claim 3, wherein the additional excipients are selected from the group consisting of disintegrants, lubricants, glidants, binders, diluents, and combinations thereof.

6. The process according to claim 1, wherein the additional excipients are selected among colloidal silicon dioxide, starch, magnesium stearate, sodium stearyl fumarate, croscarmellose sodium, crospovidone, sodium starch glycolate and talc.

7. The process according to claim 1, wherein the dry blend further comprises sucralose.

8. The process according to claim 1, wherein the compaction step is performed using a roll compactor.

9. The process according to claim 1, wherein the compression step is performed suing a rotating press.

10. The process according to claim 1, wherein the compaction step is performed at a pressure within the range of 2 to 4 Mpa.

11. The process according to claim 1, wherein the compaction is performed at a powder flow of 5 to 20 g/min.

12-21. (canceled)

22. The process according to claim 1, wherein the tablets are of a chewable type.

23. The process according to claim 1, wherein the tablets comprise 1 to 20 mg of montelukast or a pharmaceutically acceptable salt thereof.

24. The process according to claim 5, wherein:
the binder are any selected among acacia, alginic acid, carbomer, sodium carboxymethylcellulose, dextrin, ethylcellulose, gelatine, glucose, guar gum, hydroxypropylcellulose, maltose, methylcellulose, povidone, polyvinylpyrrolidone, starch, methylcellulose or polyethylene oxide;
the diluents are any selected among microcrystalline cellulose, calcium, phosphate or sulfate carbonates, dextrates, dextrins, dextrose excipients, fructose, kaolin, lactitol, anhydrous lactose, lactose monohydrate, maltose, mannitol, sorbitol, sucrose, starch, pregelatinized starch, or talc;
the glidants are any selected among: colloidal silicon, magnesium trisilicate, starch, talc or tribasic calcium phosphate; and
the disintegrants are any selected among: alginic acid, croscarmellose sodium, crospovidone, potassium polacrilin, sodium starch glycolate, and starch.

25. The process according to claim 1, wherein the tablet has the following composition:

| | | |
|---|---|---|
| Montelukast sodium | 5.20 | mg |
| Hydroxypropyl cellulose | 35.00 | mg |
| Microcrystalline cellulose | 10.00 | mg |
| Mannitol | 195.00 | mg |
| Flavour | 3.60 | mg |
| Iron oxide red | 0.10 | mg |
| Sodium stearyl fumarate | 3.50 | mg |
| Sucralose | 0.50 | mg |
| Aerosil | 0.90 | mg |
| (AcDiSol) Crossed linked sodium | 10.00 | mg |
| carboxymethyl cellulose | | |
| Sodium stearyl fumarate | 2.80 | mg |
| Total | 266.60 | mg |

26. The process according to claim 1, wherein the tablet has the following composition:

| | | | |
|---|---|---|---|
| Tablet | Montelukast sodium | 10.40 | mg |
| | Hydroxypropyl cellulose | 20.00 | mg |
| | Microcrystalline cellulose | 10.00 | mg |
| | Mannitol | 120.00 | mg |
| | Aerosil | 0.80 | mg |
| | (AcDiSol) Crossed linked sodium | 20.00 | mg |
| | carboxymethyl cellulose | | |
| | Sodium stearyl fumarate | 1.80 | mg |
| | Total | 185.00 | mg |
| Coating | Opadry II 85 F white | 5.50 | mg |
| | |
|---|---|
| Total | 190.50 |

27. The process of claim 1, wherein the tablet comprises montelukast or a salt thereof, microcrystalline cellulose and sucralose, wherein the amount of impurity does not increase by more than 0.4% by weight from the initial amount of montelukast after storage at about 40° C. and about 75% relative humidity for 1 month.

28. The process of claim 27, wherein the amount of impurity does not increase by more than 0.2%.

29. The process of claim 1 for preparing a medicament for treating or preventing a leukotriene induced condition.

30. The process according to claim 28, wherein the leukotriene induced condition is asthma or chronic bronchitis.
